(19) United States
(12) Patent Application Publication
(10) Pub. No.: US 2011/0212489 A1
(43) Pub. Date: Sep. 1, 2011

(54) CULTURE MEDIUM FOR EUKARYOTIC CELLS

(75) Inventors: Mireille Maria Gadellaa, Bemmel (NL); Edward Allen Hunter, Hancock, NY (US)

(73) Assignee: Campina Nederland Holding B.V.

(21) Appl. No.: 12/671,857

(22) PCT Filed: Aug. 4, 2008
(86) PCT No.: PCT/NL2008/050534

§ 371 Date: May 16, 2011

(30) Foreign Application Priority Data
Aug. 3, 2007 (EP) 07113813.5

(51) Int. Cl. C12P 21/00; C12N 5/07

(52) U.S. Cl. 435/70.1; 435/404; 435/405; 435/348

(57) ABSTRACT

A proteolysate of a seed material derived from a plant species of the Asteraceae family, such as sunflower, has improved properties as a constituent of a culture medium for culturing eukaryotic, in particular animal cells. The seed material is defatted and is hydrolysed to a degree of 10-50% and subsequently separated from insolubles. The cells are particularly cultured for producing desired protein products.

### FIELD OF THE INVENTION

The invention relates to the production of a protein source for an eukaryotic, in particular animal cell culture medium, as well as to cell culture medium thus produced and its use for in vitro cultivation of eukaryotic, in particular animals cells.

### BACKGROUND

The production of valuable biochemicals and biopharmaceuticals, for instance antibodies and antibiotics, by culturing mammalian, plant or insect cells requires proper culture media. Cell culture media formulations have been supplemented with a range of additives, including undefined components like fetal calf serum (FCS), several animal-derived proteins and/or protein hydrolysates of bovine origin.

Serum or serum-derived substances, such as albumin, transferrin or insulin, which are used in animal cell culture, may contain unwanted agents that can contaminate the cultures and the biopharmaceutical products obtained from these. Moreover, bovine derived protein products like hydrolysed milk proteins or bovine meat or collagen hydrolysates bear the risk of BSE contamination. Furthermore, additives derived from human serum have to be tested for all known viruses, including hepatitis and HIV, that can be transmitted by serum.

In conclusion, all serum-derived products can be contaminated by unknown agents. In the case of serum or protein additives that are derived from human or other animal sources in cell culture, numerous problems (e.g. the varying quality and composition of different batches and the risk of contamination with viruses, mycoplasma or BSE) can occur. Therefore, plant protein hydrolysates or plant peptones are commonly used in culture media that should be free of animal components.

However, growth of animal cells in media without animal-derived cell culture additives is not always satisfactory. It is frequently observed that animal cells which are cultivated in vitro grow in lumps. This is considered to be a suboptimal condition as the cells in the core of the lump are deprived of nutrients and will die. There is also a risk of clogging the tubing or the filters during downstream processing. The reduced viability of the cells can also be assessed by their appearance. Cells having a reduced viability show an irregular shape, i.e. a not-round shape, and in addition have a “granulated” cell content which is in contrast to healthy cells that have a perfectly bright, transparent cell content.

US 2004/0185561, contents herein incorporated by reference, describes the use of non-hydrolysed extracts from various plant seeds, including peanut, almond, hazelnut, corn, soybean, sunflower, etc. as a supplement in cell culture media. It is concluded therein that peanut extracts are the most effective in proliferating animal cells, especially a combination of peanut extract and potato extract. It is also concluded in US 2004/185561 that high molecular fractions (greater than 100,000 Da) are responsible for the proliferation activity of the plant seed extracts.

Ikram-U1-Haq et al. (Proceedings of Pakistan Congress of Zoology 24 (2004) 67-75), contents herein incorporated by reference, describe optimisation of submerged fermentation parameters for the production of neutral protease by Bacillus subtilis using non-hydrolysed sunflower meal.

Postemsky et al. (Micologia Aplicada Int. 18 (2006) 7-12), contents herein incorporated by reference, investigate the effect of temperature, pH and millet and sunflower seed hulls on agar medium and the effect of temperature and sunflower seed broth on liquid medium for growing certain mushrooms. The sunflower seed is neither defatted nor hydrolysed.

WO 2006/123926, contents herein incorporated by reference, relates to a peptide composition for growing and/or culturing micro-organisms and/or cells on the basis of at least one vegetable protein source, preferably from rapeseed, wheat or caraway. The effect of wheat hydrolysate is addressed in the examples.

WO 2006/128764, contents herein incorporated by reference, discloses a process for cultivating mammalian cells producing complex proteins, wherein one or more plant-derived peptones are fed to the cell culture. Plant sources soy, cotton seed and pea are exemplified. The effect of soybean hydrolysate on cultivation of CHO cells is shown in the accompanying examples.

WO 98/08934, contents herein incorporated by reference, discloses cell serum-free culture media containing polyanionic compounds such as dextran sulphate, and optionally certain peptides, such as rice peptides or soy peptides.

Parrado et al. (Process Biochemistry 28 (1993) 109-113), contents herein incorporated by reference, describe the use of sunflower meal hydrolysates as a nitrogen source for the growth of Streptococcus equisimilis producing streptokinase. The hydrolysate was obtained by Kerase treatment and had a degree of hydrolysis of 18.8%. It contained a high level of polyphenols. It was concluded that the utility of sunflower meal peptone as a nitrogen source in fermentation media is greatly restricted and that removal of polyphenols is essential.

### SUMMARY OF THE INVENTION

It was found that hydrolysates from defatted meals of Asteraceae seeds, in particular sunflower seeds, are excellently suitable for culturing eukaryotic, in particular animal cells in vitro. Thus the invention provides a cell culture medium containing hydrolysates of (defatted) sunflower material, as well as a method for cultivation of animal cells in vitro using hydrolysates of (defatted) sunflower material hydrolysates as a medium constituent. It was also found that, using the hydrolysates according to the invention as a medium constituent, the cells do not show lumping during cultivation and have a bright, transparent appearance.

### DETAILED DESCRIPTION OF THE INVENTION

The invention pertains to the use of a hydrolysate of a protein-containing seed material derived from a plant species of the Asteraceae family in a culture medium for culturing eukaryotic cells, in particular animal cells. The present invention also pertains to a medium for culturing eukaryotic, in particular animal cells, containing 2-80 wt. %, on a dry weight basis, of a hydrolysate of a seed material derived from a plant species of the Asteraceae family.

The seed material to be used according to the invention is preferably defatted. Prior to defatting, its protein content is at least 5 wt. % on dry matter, preferably at least 10 wt. % on dry matter. After defatting, the protein content is at least 10 wt. %, preferably at least 20 wt. % on dry matter basis. The seed material is derived from plant material of a species of the Asteraceae family. In particular, the plant material is from a plant used for the production of oils, such as the genera Helianthus (sunflower), especially H. annuus, Carthamus (safflower), especially C. tinctorius, Vernonia (ironweed), especially V. galamensis, Cynara (cardoon, artichoke), Guizotia (niger), Crepis (hawksbeard) etc. Preferably the plant is a species of the genus Helianthus, such as H. annuus, H. argophilus, etc. Where reference is made to species of the Asteraceae family, preference is given to species of the Helianthus, Carthamus and Vernonia genera, more particularly to the species thereof given above.

The seed material can be defatted by conventional methods such as by pressing and/or extracting the dehulled seeds using organic solvents such as hexane. Prior to fat extraction, the seeds can be dehulled and/or crushed, but this is not a prerequisite. Preferably the defatted seed material comprises protein. More preferably the defatted seed material contains at least 10 wt. % protein, more preferably at least 20 wt %. The defatted seed material preferably has a fat content of less than 10 wt. %.

A hydrolysate as used herein denotes a hydrolysate resulting from enzymatic proteolysis and can also be referred to as proteolysate. The (defatted) seed material, optionally comminuted, is subjected to hydrolysis using endo and/or exo proteases from bacterial, fungal, vegetable or animal origin or mixtures thereof; however preferably the enzyme is not from an animal source. The enzyme may be produced using recombinant DNA techniques. The preferred enzymes are endo-proteases. More preferably the enzyme comprises alkaline proteases. Most preferably, the protease is a subtilisin (Alcalase), a serine endoprotease. Particularly suitable enzymes comprise Alcalase from Novozymes, and/or papain from Merck. Other suitable enzymes comprise e.g. Neutrase. Hydrolysis conditions comprise a reaction time of between 30 minutes and 8 hours; preferably 1-6 hours, most preferably 2-4 hours; temperatures are between 20 and 65° C., preferably between 40° C. and 60° C. The pH may be adjusted between 6.0 and 8.5, preferably 6.6 and 8.0, most preferred is 7.0-8.0. The concentration of the protein to be hydrolysed in solution is between 1 and 10% protein, preferably 2-8, most preferably 3-6 wt. %. The amount of enzyme used is, based on substrate, between 0.5-10 wt %, preferably 1-5 wt %, most preferably 1.5-3.5 wt %.

The hydrolysis is preferably performed until a degree of hydrolysis of between 5 and 50%, preferably between 10 and 40%, most preferably between 10 and 30%, is attained. The hydrolysis reaction is terminated using a heat treatment. Preferably, the heat treatment encompasses a heating time of between 15 and 90 minutes between 80 and 100° C. (batch heat treatment), or 1-5 minutes at 100-120° C. (High Temperature Short Time treatment, HTST). Degree of hydrolysis may be determined using conventional formol titration, as demonstrated in the examples. After termination of the hydrolysis reaction, the reaction mixture can optionally be polished to remove insoluble parts, for example using centrifugation or filtering aids know in the art like diatomaceous earth (e.g. Celite®, Dicalite®, Hyflo®). Preferably, the hydrolysate contains less than 10 wt. %, on dry matter basis, of water-insoluble material, more preferably less than 5 wt. %, most preferably less than 2 wt. %. The hydrolysate can be dried by spray drying or freeze drying. The hydrolysate may be used as such or may be further fractionated.

The hydrolysate preferably contains between 20 and 80 wt. %, especially between 20 and 60 wt. % of peptides having a molecular weight of 100-500 Da and/or between 10 and 30 wt. % of peptides of a molecular weight between 500 an 1000 Da on total protein basis. In terms of peptide length, the hydrolysate preferably contains at least 15 wt. %, more preferably at least 25 wt. %, most preferably at least 35 wt. %, up to e.g. 85 wt. %, more preferably up to 65 wt. %, most preferably up to 55 wt. % of di- to penta-peptides, between 8 and 30 wt. % of hexa- to nonapeptides, at least 8 w. %, especially between 15 and 60 wt. % of higher peptides and between 0.1 and 30 wt. %, preferably between 0.5 and 10 wt. % of free amino acids, on total protein basis. In a preferred embodiment, the hydrolysate may be ultrafiltered, preferably using a 5 or 10 kD molecular weight cut-off. The hydrolysate may contain further constituents such as carbohydrates, soluble fibres, multivalent metal salts, etc. Preferably the protein content (all proteinaceous material including free amino acids) is between 30 and 90 wt. %, more preferably between 45 and 85 wt. %. These amounts are on a dry weight basis.

The hydrolysate may be combined with other conventional constituents of culture media such as plant or animal cytokines and/or growth factors (provided that these are not of animal origin), vitamins, minerals, amino acids, buffering salts, trace elements, nucleosides, nucleotides, phytohormones, sugars including glucose, antibiotics and the like. Phytohormones comprise auxins, gibberellins, abscisic acid and combinations thereof.

Also commercially available basal media may be used in combination with the hydrolysate of the invention. For an animal cell line as CHO-1, Power CHO-1 CD from Lonza, IS CHO-CD from Irvine Scientific, or Excell 325 PF CHO from SAFC may be used. For plant cells, Murashige and Skoog basal medium obtainable from SAFC may be used. The hydrolysate may also be combined with other protein sources, such as hydrolysates from wheat, soy and pea, e.g. in a weight ratio of Asteraceae seed protein to other protein between 9:1 and 1:4, preferably between 4:1 and 1:2, on protein basis.

The cell culture medium and the method of culturing both according to the invention are capable of supporting cultivation of eukaryotic, in particular animal cells, which capability means that it enables at least the survival, proliferation and/or differentiation of—and preferably also the expression of product by—the cells in vitro. Cultivation in batch, fed batch, continuous or perfusion reactors are all envisaged.

Cell growth curves can be separated in a real growth phase in which the cells multiply and grow, and a production phase, in which the cells are more or less in a steady state, but start to produce the metabolites of interest, e.g. antibodies. The hydrolysate of the invention is suitable for supporting both the growth phase and the production phase of animal or other eukaryotic cells.

The cell culture medium may be provided as a liquid or in a powdered, dried form. The amount of (essentially water-soluble) hydrolysate in the liquid medium can be determined by the skilled person, but comprises preferably 0.01-4.0 wt/vol %, more preferably 0.05-2.0 wt/vol %, or 0.05-1.0 wt/vol %, even more preferably 0.1-1.0 wt/vol %, and most preferably 0.2-0.6 wt/vol %.

The amount of hydrolysate in a dry culture medium that can be reconstituted with water is depending on the medium components, but is typically in the range of 2-80% w/w, preferably 5-50% w/w. The cell culture medium also preferably contains sugars, in particular glucose, preferably in a dry weight ratio of glucose to hydrolysate between 10 and 0.1, more preferably between 2.5 and 0.4, and further constituents as described above.

Furthermore, the invention concerns the use of the cell medium for culturing eukaryotic cells. Eukaryotes comprise Fungi (including yeasts), Protista, Chromista, Plantae and Metazoa (animals). The invention especially concerns the use for culturing plant cells, for example rice, tobacco and maize, and in particular animal cells, preferably in vitro cultivation. The cells to be cultured may be from a natural source or may be genetically modified. Animal cells especially comprise vertebrate and invertebrate cells, including mammalian cells such as human cells e.g. PER C6 Cells®, rodent cells, in particular Chinese Hamster Ovary (CHO) cells, avian, fish, reptile, amphibian or insect cells.

The cells cultured by the method of the invention are in particular used for expression of protein products that may be further purified in biopharmaceutical industry. Non-limiting examples of protein products that can advantageously be produced in the culture medium of the invention include erythropoietin (for treating blood disorders), etanercept (TNF-α inhibitor for treating rheumatic diseases and gout), alpha dornase (deoxyribonuclease for the treatment of cystic fibrosis), beta-interferon (for treating multiple sclerosis) and a wide range of therapeutic monoclonal antibodies. The desired protein products may be recovered by methods known in the art, such as separating the cells from the culture medium and isolating the protein products from the cell-free liquid (supernatant) e.g. by fractionation, affinity chromatography (adsorption-desorption) or the like, or combinations thereof.

Furthermore, the invention concerns a kit comprising a hydrolysate of protein-containing seed material of a plant species of the Asteraceae family, and one or more constituents of culture media selected from plant or animal cytokines and/or growth factors, vitamins, minerals, amino acids, buffering salts, trace elements, nucleosides, phytohormones, nucleotides, sugars and antibiotics. The constituents may be present in the kit as one or more combinations. For example, the protein-containing hydrolysate may be separately present in dry or dissolved form and part or all of the further constituents of culture media such as plant or animal cytokines and/or growth factors, vitamins, minerals, amino acids, buffering salts, trace elements, nucleosides, nucleotides, phytohormones, sugars and antibiotics, may be present as a separate combination. Alternatively, the hydrolysate may be premixed with e.g. the (additional, i.e. not Asteraceae-derived) amino acids and/or sugars, and any remaining constituents may be present separately or in one or more combinations. It is preferred that at least one of the compositions is a liquid, which liquid may advantageously be sterilised. The compositions of the kit are mixed prior to use of the culture medium.

It has thus been found that the hydrolysate according to the invention and its use have several important advantages. Firstly, animal cells that are cultured in vitro are not growing in lumps or clusters but are present as single cells. Secondly, the viability of the cells is excellent as judged by their perfect round shape and bright transparent cell content. Thirdly, much higher cell densities can be obtained compared to state of the art cell culture media such as those based on non-serum protein, in particular soy protein, without compromising the expression level of the desired cell products. Fourthly, the hydrolysate can be combined with any basal culture medium for in vitro cultivation of animal cells, enabling the manufacture of a wide variety of cell culture media with the advantages mentioned above. Also the cultivation can be extended over prolonged periods, resulting in higher product yields.

### DESCRIPTION OF THE FIGURES

FIG. 1 shows the growth performance of CHO cells (CRL 11397) in IS CHO-CD medium containing 0.4% w/v hydrolysate.

FIG. 2: Growth performance CHO cells (CRL 11397) in various media with and without 0.4% sunflower hydrolysate. The solid lines represent the standard media with the sunflower hydrolysate S1 and the broken lines represent the standard media without the sunflower hydrolysate.

### EXAMPLES

### Example 1a

### Preparation of Sunflower Protein Seed Hydrolysate

200 g defatted sunflower meal from ADM (35% wt protein) was dispersed in 1.8 kg water at 60° C. to make a 10% solution. The pH of the solution was adjusted to 7.5±0.1 with 50% sodium hydroxide. To this solution, 5.05 g (2.5% on solids) of Alcalase 2.4 L (NOVOZYMES, USA) was added. The pH was allowed to drift during the hydrolysis. After 4 hours of hydrolysis at 60° C., the pH of the solution was adjusted to 6.7±0.1 with 50% sodium hydroxide and the solution was batch inactivated at 95° C. for 30 min. The solution was cooled to 60° C. and approximately 100 g of Hyflo diatomaceous earth (DE) was added and the solution was filtered using a Buchi vacuum filtration funnel and filter paper. After filtration the solution was freeze dried. This hydrolysate is named S1.

### Analysis of S1

Table 1 shows the molecular weight distribution as analysed with a Superdex peptide column (Amersham Biosciences). The column was calibrated with protein markers with a known molecular weight.

Nitrogen content and degree of hydrolysis were determined using formol titration. The total nitrogen (TN) content of the hydrolysate was measured to be 9.4%, which results in a total protein content of 53.6% using a nitrogen conversion factor of 5.7.

The amino nitrogen (AN) content was 1.5%.
The degree of hydrolysis was determined to be 14% using the formula:

DH={(AN/TN)*130}−5.9.

TABLE 1
Molecular weight (Da) distribution
(%) (weight ranges approximate)

| | | 10,000- | 5000- | 2000- | 1000- | |
|---|---|---|---|---|---|---|
| | >10,000 | 5000 | 2000 | 1000 | 500 | <500 |
| S1 | 16 | 3 | 6 | 10 | 18 | 47 |
| S2 | 0 | 0 | 2 | 6 | 14 | 78 |
| S3 | 1 | 1 | 4 | 8 | 12 | 75 |
| S4 | 0 | 0 | 2 | 7 | 13 | 78 |

### Example 1b

A solution of 10% solids was made with high protein sunflower meal (High Protein Sunflower Pellets, 37% total protein, of Glencore International), at 60° C. in a water bath. The slurry was heat treated at 80° C. for 10 minutes. Then it was cooled to 60° C. and the pH was measured. Sodium hydroxide was used to adjust the pH to 7.5±0.1. Alcalase enzyme was added to the mixture at 4% concentration on solids base and hydrolysis was carried out for 6 hours at 60° C. After 6 hours, the slurry was heat inactivated at 95° C. for 30 minutes. The hydrolysed mixture was vacuum-filtered to remove the coarse impurities. The slurry was then ultra-filtered using a Koch HFK-131 spiral wound membrane device having a cut-off of 10,000 Da, and spray dried to obtain a powdered hydrolysate. The degree of hydrolysis obtained in this example was 36%. This hydrolysate is denoted as S2.

### Example 1c

A procedure similar to Example 1b was performed except that the enzyme papain from Merck was used at 2% concentration on solids base. The mixture was hydrolysed for 3 hours and then heat inactivated at 95° C. for 30 minutes. The degree of hydrolysis obtained in this example was 25%. This hydrolysate is denoted as S3.

### Example 1d

A procedure comparable to Example 1b was performed except that low protein sunflower meal (Sunflower Pellets, having 32% total protein, of Glencore Int.), was hydrolysed using Alcalase at 2% on the solids base for 3 hours. The degree of hydrolysis obtained in this example was 42%. The hydrolysate was denoted as S4. Molecular weight distribution of S2-S4 was determined according to the method of Example 1a and summarized in Table 1.

### Example 2a

### In Vitro Cultivation of Cho Cells

### Medium Preparation

To various commercially available media, sunflower hydrolysate (S1) at a concentration of 0.4 (w/v) % was added.

The following media were tested: Power CHO-1 CD medium (Lonza, Cat. No. 12-770 Q), IS CHO-CD medium (Irvine Scientific, Cat. No. 91119) and Ex-cell 325 PF CHO medium (SAFC, Cat No. 14340C). L-Glutamine (2 mM), hypoxanthine (100 μM) and thymidine (15 μM) were added if not already present in the medium. Penicillin and streptomycin were added to prevent bacterial growth.

### Cell Lines

Two different IgG expressing CHO cell lines were used (CHO-2,: ATCC CRL 11397, producing IgG4) and CHO-3, ATCC CRL 12445, producing IgG1). Before use the cell lines were adapted to animal-free culture conditions.

### Growth and Production Curves

To measure growth and production curves, CHO cells were grown in 6-well plates. To 1 ml of cell suspension 3 ml of medium was added. Chemically defined media with and without added hydrolysates were tested. Hydrolysates from soy (SE50MAF-UF; DMV International, about 50% protein content) and gluten origin (WGE80M-UF; DMV International, about 80% protein content) were tested in the same concentration in the same medium as the sunflower hydrolysate. Three times per week 2 ml medium was replaced by fresh medium.

Cells were counted manually and production of IgG was measured by ELISA before every medium change. The cells were visually inspected using a phase contrast microscope (Zeiss Axiovert 25, 400× magnification). The appearance was dramatically improved when sunflower hydrolysate was present in the medium. Only single cells were observed and no aggregation of cells was seen. This was in contrast with the observation that a lot of cell aggregates were present in CHO cell cultures grown in standard medium or in medium using other hydrolysates. Besides that the cell shape was positively affected. Cells had a much more round and bright appearance when cultured in medium containing the sunflower hydrolysate.

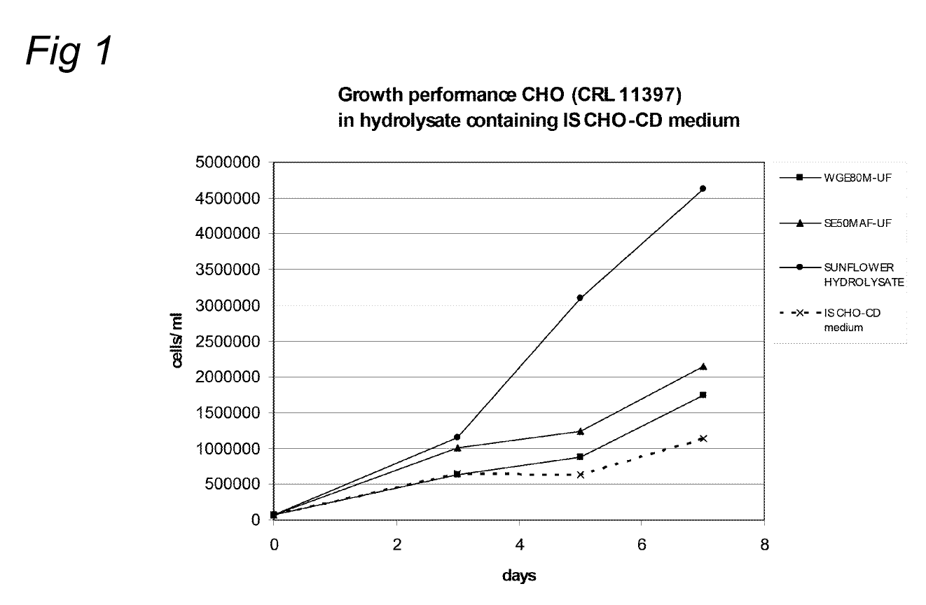

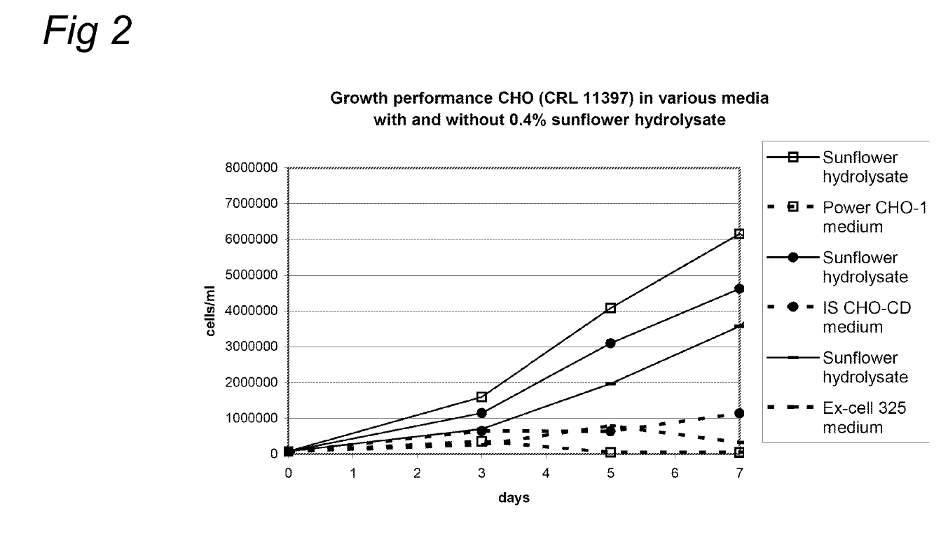

Table 2 and FIGS. 1 and 2 show the growth performance of CHO cells in the presence of various media with and without various plant protein hydrolysates.

TABLE 2
Growth performance of CHO (CRL 11397) cells in
IS CHO-CD medium with and without hydrolysates.

| | Cell counts (times 100,000 cells) | | | |
|---|---|---|---|---|
| IS CHO-CD medium with: | Day 0 | Day 3 | Day 5 | Day 7 |
| WGE80M-UF | 0.75 | 0.63 | 0.87 | 1.75 |
| SE50MAF-UF | 0.75 | 1.00 | 1.23 | 2.15 |
| SUNFLOWER | 0.75 | 1.15 | 3.10 | 4.62 |
| HYDROLYSATE S1 | | | | |
| (no addition of hydrolysate) | 0.75 | 0.64 | 0.64 | 1.14 |

All cell counts are * 10⁵ cells

### Example 2b

Cell growth experiments were carried out using the sunflower seed hydrolysates S2-S4, and using CHO-2 cells (ATCC CRL 11397) essentially according to Example 2a. The basal growth medium used was Power CHO. The amount of hydrolysates added to the medium was 0.2% w/w. As a control, Power CHO without hydrolysate was used. As a reference, Power CHO including a soy hydrolysate (0.4% w/w SE50MAF-UF, DMV International) was used.

In this experiment, cell counting was performed using a Cedex HiRes instrument (Innovatis, Germany). Cedex HiRes uses the Trypan Blue exclusion method to determine the cell concentration and viability within cell cultivation processes. It stains the samples with Trypan Blue and analyses the images generated with scanner technology and eventually presents the cell count in a few minutes. The following protocol was used:

The CHO-cell suspension in 6-well plates was re-suspended to ensure homogeneity. Subsequently, a representative sample of 150 μL was transferred to the sample cup. 150 μL of Dulbecco's Phosphate Buffered Saline (Invitrogen, Cat. No. 14190-094) was added to the sample cup to get a dilution factor of 1:1. The sample cups were placed in a multi-sample tray. Desired sample data and the parameters were defined for each sample. The comments were entered and the status of all the samples was activated to start the measurements. Results obtained were printed and exported to Excel for further calculations
The results of the cell counts are presented in Table 3:

TABLE 3
Growth performance of CHO-2 cell in Power
CHO medium with sunflower hydrolysate; with
soy hydrolysate and without hydrolysates.

| | Day | | | | | | |
|---|---|---|---|---|---|---|---|
| Samples | 0 | 2 | 4 | 7 | 9 | 11 | 13 |
| S2 | 1.00 | 1.99 | 4.74 | 9.41 | 12.20 | 12.66 | 19.28 |
| S4 | 1.00 | 2.03 | 5.71 | 10.75 | 10.53 | 10.44 | 11.41 |
| S3 | 1.00 | 1.62 | 4.52 | 6.24 | 5.87 | 8.68 | 10.87 |
| SE50MAF-UF | 1.00 | 1.85 | 4.86 | 4.87 | 5.22 | 6.26 | 9.62 |
| Control (no | 1.00 | 1.27 | 1.77 | 3.47 | 4.74 | 4.76 | 5.75 |
| hydrolysate) | | | | | | | |

All cell counts are * 10⁵ cells/ml

### Determination of Cell Viability.

Using the same Cedex HiRes instrument, viability of the cultured cells was determined as well. Table 4 shows the percentage of viable cells in various stages of cell growth.

TABLE 4
Viability of CHO-2 cells grown in Power
CHO with and without hydrolysates

| | Days | | |
|---|---|---|---|
| Samples | 0 | 3 | 5 |
| S2 | 75.1 | 83.9 | 85.1 |
| SE50MAF-UF | 75.1 | 28.4 | 57.1 |
| Control (Power CHO without hydrolysates) | 75.1 | 20.4 | 56.2 |

The obtained growth curves indicate that the cells can be cultured using the sunflower hydrolysate. Cell density is higher than the density obtained using the other tested media.

Sunflower hydrolysate gives a good growth and production when added to various media. It was observed that very good production of IgG4 took place when CHO-2 cells were grown in a culture medium supplemented with the sunflower hydrolysates S2, S3 or S4. This indicates that the sunflower hydrolysate can be used in different processes.

The most remarkable observation was that the CHO cells are much better shaped when grown in medium containing the sunflower hydrolysate. The cells looked perfectly round and had a bright appearance, which was considered an indication for the cell viability. There were only single cells in the culture, no clusters at all. In suspension cultures clusters/clogs are very common but not desirable. Cells in the core of big clusters will die and there is a risk of clogging the tubing or the filters during downstream processing.

1.-15. (canceled)

16. A culture medium for culturing eukaryotic cells comprising a hydrolysate of a protein-containing seed material derived from a plant species of the genus Helianthus, Carthamus or Vernonia.

17. The culture medium according to claim 16, wherein the plant species is from the genus Helianthus.

18. The culture medium according to claim 17, wherein the plant species is Heliantus annuus.

19. The culture medium according to claim 16, wherein the seed material is defatted prior to hydrolysis.

20. The culture medium according to claim 16, wherein the hydrolysate has a degree of hydrolysis of between 5 and 50%.

21. The culture medium according to claim 16, wherein the eukaryotic cells comprise animal cells.

22. A culture medium for culturing eukaryotic cells comprising:
(a) 2-80 wt. %, on a dry weight basis, of a hydrolysate of a defatted seed material derived from a plant species of the genus Helianthus, Carthamus or Vernonia; and
(b) at least one cytokine, growth factor, or both.

23. The culture medium according to claim 22, wherein the culture medium is a liquid and comprises 0.01-4.0 wt/vol % of the hydrolysate.

24. The culture medium according to claim 22, further comprising vitamins, minerals, amino acids, buffering salts, trace elements, nucleosides, nucleotides, phytohormones, sugars, antibiotics, protein hydrolysates other than a hydrolysate of a defatted seed material derived from a plant species of the genus Helianthus, Carthamus or Vernonia.

25. A liquid culture medium for culturing eukaryotic cells comprising 0.05-1.0 wt/vol % of a hydrolysate of a defatted seed material derived from a plant species of the genus Helianthus, Carthamus or Vernonia.

26. The liquid culture medium according to claim 25, comprising 0.1-0.6 wt/vol % of the hydrolysate of a defatted seed material.

27. A method of culturing eukaryotic cells in vitro, comprising growing the eukaryotic cells in a culture medium comprising a hydrolysate of a seed material derived from a plant species of the genus Helianthus, Carthamus or Vernonia.

28. The method according to claim 27, in which the eukaryotic cells comprise animal cells.

29. The method according to claim 27, in which the animal cells comprise mammalian cells, insect cells, or both.

30. The method according to claim 27, in which the culture medium further comprises a hydrolysate from wheat, soy, or pea protein.

31. A method for producing a protein product, the method comprising:
(a) culturing eukaryotic cells producing the protein product in a culture medium comprising a hydrolysate of a seed material derived from a plant species of the genus Helianthus, Carthamus or Vernonia; and
(b) recovering the protein product from the culture medium.

32. A kit for eukaryotic cell culture comprising:
(a) a composition comprising a hydrolysate of protein-containing seed material of a plant species of the genus Helianthus, Carthamus or Vernonia, and
(b) a composition comprising cytokines, growth factors, vitamins, minerals, amino acids, buffering salts, trace elements, nucleosides, nucleotides, phytohormones, sugars, antibiotics, or combinations thereof,
wherein the compositions are to be mixed prior to use.

33. The kit according to claim 32, in which at least one of the compositions is a liquid, which liquid is optionally sterilised.
